(19) United States
(12) Patent Application Publication
(10) Pub. No.: US 2022/0395400 A1
(43) Pub. Date: Dec. 15, 2022

(54) Dressings With Polymer Delivery

(71) Applicant: KCI Licensing, Inc., San Antonio, TX (US)

(72) Inventors: Justin Alexander LONG, Lago Vista, TX (US); Timothy Mark ROBINSON, Shillingstone (GB); Christopher Brian LOCKE, San Antonio, TX (US); Thomas Alan EDWARDS, Hampshire (GB)

(21) Appl. No.: 17/621,560

(22) PCT Filed: May 26, 2020
(86) PCT No.: PCT/US2020/034562

§ 371 Date: Dec. 21, 2021

Related U.S. Application Data
(60) Provisional application No. 62/867,986, filed on Jun. 28, 2019.

(51) Int. Cl. A61F 13/00; A61F 13/02; A61L 15/42; A61L 15/22; A61M 1/00

(52) U.S. Cl. CPC A61F 13/00063; A61F 13/0216; A61L 15/425; A61L 15/225; A61M 1/915; A61F 2013/00604

(57) ABSTRACT

Dressings and kits for use in wound therapy and negative-pressure therapy comprising a manifold layer comprising porous open-cell liquid permeable foam and a polymer composition The polymer composition comprises a polymer and an active agent, such as collagen and oxidized regenerated cellulose. The foam may be felted. Methods of making and using the dressings are also provided.

### RELATED APPLICATIONS

The present application claims priority to U.S. Provisional Patent Application No. 62/867,986, entitled “Dressings With Polymer Delivery,” filed Jun. 28, 2019, which is incorporated herein by reference for all purposes.

### TECHNICAL FIELD

The claimed subject matter relates generally to treatment of tissue, including without limitation compositions, dressings, and other apparatuses for application to a tissue site, such as a wound.

### BACKGROUND

A wide variety of materials and devices, generally characterized as “dressings,” are generally known in the art for use in treating an injury, defect, or other disruption of tissue. Such disruptions of tissue may be the result of trauma, surgery, or disease, and may affect skin or other tissues. In general, dressings may control bleeding, absorb exudate, ease pain, assist in debriding tissue, protect tissue from infection, or otherwise promote healing and protect tissue from further damage.

Some dressings may protect tissue from, or even assist in the treatment of, infections associated with wounds. Infections can retard wound healing and, if untreated, can result in tissue loss, systemic infections, septic shock and death. While the benefits of dressings are widely accepted, improvements to dressings may benefit healthcare providers and patients.

### BRIEF SUMMARY

New and useful compositions, apparatuses, systems, and methods for treating tissue are set forth in the appended claims. Illustrative embodiments are also provided to enable a person skilled in the art to make and use the claimed subject matter.

For example, a dressing for treating a tissue site, optionally with negative pressure, is described. In various embodiments, a dressing can comprise a manifold layer having a porous open-cell liquid permeable foam and a polymer composition bound to the foam. The foam may comprise 50-150 micron-sized pores that are capable of distributing, in some embodiments, negative pressure to the tissue site and withdrawing tissue exudate. The polymer may comprise an active agent and a polymer carrier, wherein the polymer carrier is capable of releasing the active agent when exposed to tissue exudate.

In various embodiments, the foam has a first side configured to be adjacent to the tissue site and a second side opposite to the first side. The polymer composition may be present (e.g., bound) on the first side, the second side, or both the first side and second side of the foam. The size of the pores of the foam may be determined by a measurement normal to the first side or the second side of the foam. In some embodiments, the foam has more than 350 pores per linear inch (ppi) as determined by a measurement normal to the first side or the second side of the foam.

The foam may comprise a felted foam, and may exhibit a firmness factor (which may be determined as discussed herein). In some embodiments, the foam has a firmness factor of four to six. In some embodiments, the foam has a firmness factor of five.

In various embodiments, the foam is an open-cell foam and/or a reticulated foam. The foam may be a polymer foam, such as acrylic, polyurethane, polyolefin, polyethylene, polyacetate, polyamide, polyester, polyether, polyether block amide, thermoplastic vulcanizate, polyvinyl alcohol foams, or combinations thereof. In some embodiments, at least a portion of the foam is a plasma or corona treated foam that increases the hydrophilicity of the treated portion of the foam as compared to the same foam that has not been treated.

In some embodiments, the biocompatible polymer may comprise an active agent, such as collagen, oxidized regenerated cellulose (ORC), or a combination thereof. In some embodiments, the active agent, such as collagen and ORC, may be encapsulated in a carrier. The carrier may comprise a water-soluble or a water-sensitive polymer.

In various embodiments, the manifold layer has a thickness of from 2 mm to 8 mm or from 3 mm to 5 mm. The manifold layer may further comprise an absorbent material, particularly a super absorbent material.

In further embodiments, the dressing comprises a cover configured to be disposed adjacent to the manifold layer and to form a seal around the tissue site. The cover may comprise a channel configured to distribute negative pressure.

In some embodiments, the dressing further comprises a fluid-control layer configured to be disposed between the manifold layer and the tissue site. The fluid-control layer can have a plurality of fluid restrictions with a uniform size or varied sizes. The plurality of fluid restrictions may comprise or consist essentially of a plurality of perforations, slots, fenestrations, slits, or elastomeric valves configured to permit fluid flow and inhibit exposure of the manifold layer to the tissue site.

In some embodiments, a dressing comprises a fluid permeable material comprising a plurality of pores having a first surface, a second surface, and a third surface extending between the first and second surfaces, wherein each of the plurality of pores has a pore size in at least one dimension at the third surface permanently smaller than the diameter of the pore at the plane of the first and/or second surfaces, for example, by felting. The dressing may further comprise a biocompatible polymer composition adhered to the fluid permeable material. The composition may comprise collagen, ORC, and a water-soluble and/or water-sensitive polymer.

The present disclosure also provides systems for treating a tissue site. For example, a system may comprise a dressing, such as any of the dressing embodiments described herein, and a coupling capable of fluidly-coupling the dressing to a negative-pressure source. The system may further comprise a fluid container fluidly coupled between the dressing and the negative-pressure source. The system may further comprise a fluid source fluidly coupled to the dressing, the fluid container capable of being coupled to the negative pressure source.

The present disclosure also provides methods for treating a tissue site, comprising applying a dressing to the tissue site, wherein the dressing is any of the dressing embodiments described herein. In some embodiments, methods further comprise sealing the dressing in a void adjacent to the tissue site, wherein the sealing is configured to allow the dressing to provide negative pressure to the tissue site, and fluidly coupling the dressing to a negative-pressure source. The method may further comprise applying negative pressure from the negative-pressure source to the dressing. In some embodiments, the dressing may comprise a tissue contact layer contacting the tissue site during the applying of the negative pressure. The method may further comprise fluidly coupling a fluid container between the dressing and the negative-pressure source and transferring exudate from the dressing to the fluid container. The method may further comprise delivering a fluid from a fluid source through the dressing.

The present disclosure also provides methods for treating a tissue site, comprising applying a dressing to the tissue site, wherein such methods do not comprise applying negative pressure to the dressing. For example, such methods including a method of treating a wound with an active agent, such as collagen, ORC and/or other active agent, comprising applying a dressing according to any of the dressing embodiments described herein. In some embodiments, the dressing comprises a wound contact layer in direct contact with the wound.

The present disclosure also provides methods for making a dressing, including any of the dressing embodiments described herein. For example, a method for making a dressing may comprise providing a porous open-cell liquid permeable form or a fluid permeable material having a first firmness and a first thickness, and compressing and heating the foam to a second firmness permanently, wherein the second firmness is higher than the first firmness. The method may further comprise cutting the foam to a second thickness, wherein the second thickness is less than the first thickness. The method may further comprise attaching a polymer composition or a biocompatible polymer to the foam or fluid permeable material, such as by applying the polymer composition or the biocompatible polymer composition to the foam or fluid permeable material.

The present disclosure also provides wound therapy kits, comprising any of the dressing embodiments described herein. Such kits may further comprise instructions for use. A kit may further comprise a cover configured to cover the dressing to form a sealed therapeutic environment.

Objectives, advantages, and a preferred mode of making and using the claimed subject matter may be understood best by reference to the accompanying drawings in conjunction with the following detailed description of illustrative embodiments.

### DRAWINGS

FIG. 1 is a functional block diagram of an example embodiment of a therapy system that can provide negative-pressure treatment and instillation treatment in accordance with this specification;

FIG. 2 is a graph illustrating additional details of example pressure control modes that may be associated with some embodiments of the therapy system of FIG. 1;

FIG. 3 is a graph illustrating additional details that may be associated with another example pressure control mode in some embodiments of the therapy system of FIG. 1;

FIG. 4 is a chart illustrating details that may be associated with an example method of operating the therapy system of FIG. 1;

FIG. 5 is an assembly view of an example of the dressing 110 of FIG. 1, illustrating additional details that may be associated with some embodiments of the therapy system of FIG. 1.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

As noted above, the present disclosure provides dressings for treating a tissue site. In various embodiments, a dressing comprises a manifold layer comprising: a porous open-cell liquid-permeable foam comprising 50 to 150 micron sized pores capable of distributing negative pressure to the tissue site and withdrawing tissue exudate; and a polymer composition bound to the foam, the polymer composition comprising an active agent and a polymer carrier for the active agent, the polymer carrier capable of releasing the active agent when exposed to tissue exudate; and wherein the foam has a first side configured to be adjacent to the tissue site and a second side opposite to the first side, and the polymer composition is present on the first side or the second side or both the first and the second sides of the foam, and wherein the pore size is determined by a measurement normal to the first side or second side of the foam.

In some embodiments, the dressing comprises a fluid permeable material comprising a plurality of pores having a first surface, a second surface, and a third surface extending between the first and second surfaces, wherein each of the plurality of pores has a pore size in at least one dimension at the third surface permanently smaller than the diameter of the pore at the plane of the first and/or second surface; and a biocompatible polymer composition adhered to the fluid permeable material, the composition comprising collagen, oxidized regenerated cellulose (ORC), and a water-soluble and/or water-sensitive polymer.

Example Therapy System

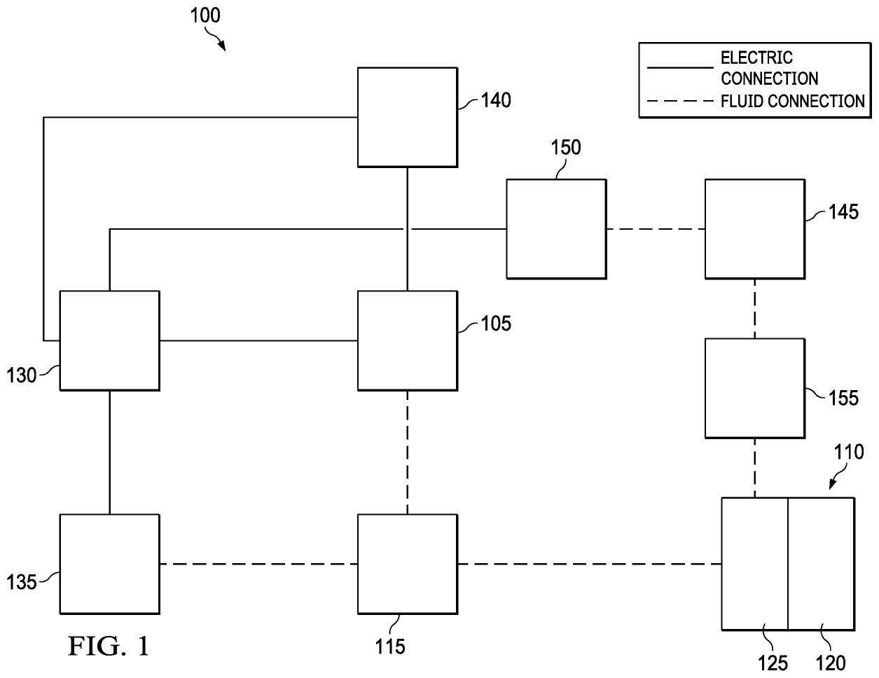

FIG. 1 is a simplified functional block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments. In some embodiments, the tissue interface comprises a fluid manifold, wherein the dressing further comprises a tissue contact layer.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Tex.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

The therapy system 100 may also include a source of instillation solution. For example, a solution source 145 may be fluidly coupled to the dressing 110, as illustrated in the example embodiment of FIG. 1. The solution source 145 may also be a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

The solution source 145 may be fluidly coupled to a positive-pressure source such as a positive-pressure source 150, a negative-pressure source such as the negative-pressure source 105, or both in some embodiments. A regulator, such as an instillation regulator 155, may also be fluidly coupled to the solution source 145 and the dressing 110 to ensure proper dosage of instillation solution (e.g. saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130, the solution source 145, and other components into a therapy unit.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The dressings disclosed herein may be used to treat a tissue site in the context of various therapies.

In some embodiments, the dressing 110 is used in negative-pressure therapy. In some embodiments, the dressing 110 disclosed herein may be used for at least 5, 6, 7, 8, 9, 10, 11 or 12 days to promote granulation and/or minimize tissue in-growth with a source of negative pressure. For example, the dressing 110 disclosed herein may remain on a tissue site, such as a surface wound, for at least 5 to 7 days.

Clinical studies and practice have shown that reducing pressure in proximity to a tissue site can augment and accelerate growth of new tissue at the tissue site. The applications of this phenomenon are numerous, but it has proven particularly advantageous for treating wounds. Treatment of tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits may increase development of granulation tissue and reduce healing times.

“Negative pressure” generally refers to a pressure less than a local ambient pressure, such as ambient pressure in a local environment external to a sealed therapeutic environment. In many cases, local ambient pressure may also be atmospheric pressure near a tissue site. Alternatively, the pressure may be less than a hydrostatic pressure associated with tissue at a tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

In operation, the dressing 110 may be positioned within, over, on, or otherwise proximate to a tissue site. Additionally as noted above, in some embodiments, the dressing 110 may include a cover, such as the cover 125, that may be sealed to an attachment surface near a tissue site. For example, the cover 125 may be sealed to undamaged epidermis peripheral to a tissue site. In some embodiments, the components of the dressing 110 may be positioned sequentially. In some other embodiments, the dressing 110 may be preassembled, for example, such that the cover 125 is positioned with respect to other components of the dressing 110 prior to placement proximate a tissue site. Thus, the cover 125 can seal any other layers of the dressing 110 in a therapeutic environment proximate to a tissue site, substantially isolated from the external environment.

Additionally, in some embodiments a therapy method may further comprise fluidly coupling a negative-pressure source to a dressing, such as the dressing 110, and operating the negative-pressure source to generate a negative pressure proximate to a tissue site. For example, the negative-pressure source 105 may be coupled to the dressing 110 such that the negative-pressure source 105 may be used to reduce the pressure beneath the cover 125. Negative pressure applied across a tissue site, for example, via the dressing 110, may be effective to induce macrostrain and microstrain at the tissue site, as well as remove exudate and other fluids from the tissue site. Exudate and other fluid may be stored in one or more layers of the dressing 110 in some embodiments. Additionally or alternatively, exudate and other fluid can be transferred to an external container, such as the container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

In some embodiments, the controller 130 may have a continuous pressure mode, in which the negative-pressure source 105 is configured to provide a constant target negative pressure for the duration of treatment or until manually deactivated. Additionally or alternatively, the controller may have an intermittent pressure mode. For example, the controller 130 can operate the negative-pressure source 105 to cycle between a target pressure and atmospheric pressure. For example, the target pressure may be set at a value of 135 mmHg for a specified period of time (e.g., 5 min), followed by a specified period of time (e.g., 2 min) of deactivation. The cycle can be repeated by activating the negative-pressure source 105, which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time may be a value substantially equal to the initial rise time.

In some example dynamic pressure control modes, the target pressure can vary with time. For example, the target pressure may vary in the form of a triangular waveform, varying between a negative pressure of 50 and 135 mmHg with a rise time set at a rate of +25 mmHg/min. and a descent time set at −25 mmHg/min. In other embodiments of the therapy system 100, the triangular waveform may vary between negative pressure of 25 and 135 mmHg with a rise time set at a rate of +30 mmHg/min and a descent time set at −30 mmHg/min.

In some embodiments, the controller 130 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 130, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

In some embodiments, the controller 130 may receive and process data, such as data related to instillation solution provided to the tissue interface 120. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second to 30 minutes. The controller 130 may also control the operation of one or more components of the therapy system 100 to instill solution. For example, the controller 130 may manage fluid distributed from the solution source 145 to the tissue interface 120. In some embodiments, fluid may be instilled to a tissue site by applying a negative pressure from the negative-pressure source 105 to reduce the pressure at the tissue site, drawing solution into the tissue interface 120. In some embodiments, solution may be instilled to a tissue site by applying a positive pressure from the positive-pressure source 150 to move solution from the solution source 145 to the tissue interface 120. Additionally or alternatively, the solution source 145 may be elevated to a height sufficient to allow gravity to move solution into the tissue interface 120.

The controller 130 may also control the fluid dynamics of instillation by providing a continuous flow of solution or an intermittent flow of solution. Negative pressure may be applied to provide either continuous flow or intermittent flow of solution. The application of negative pressure may be implemented to provide a continuous pressure mode of operation to achieve a continuous flow rate of instillation solution through the tissue interface 120, or it may be implemented to provide a dynamic pressure mode of operation to vary the flow rate of instillation solution through the tissue interface 120. Alternatively, the application of negative pressure may be implemented to provide an intermittent mode of operation to allow instillation solution to dwell at the tissue interface 120. In an intermittent mode, a specific fill volume and dwell time may be provided depending, for example, on the type of tissue site being treated and the type of dressing being utilized. After or during instillation of solution, negative-pressure treatment may be applied. The controller 130 may be utilized to select a mode of operation and the duration of the negative pressure treatment before commencing another instillation cycle.

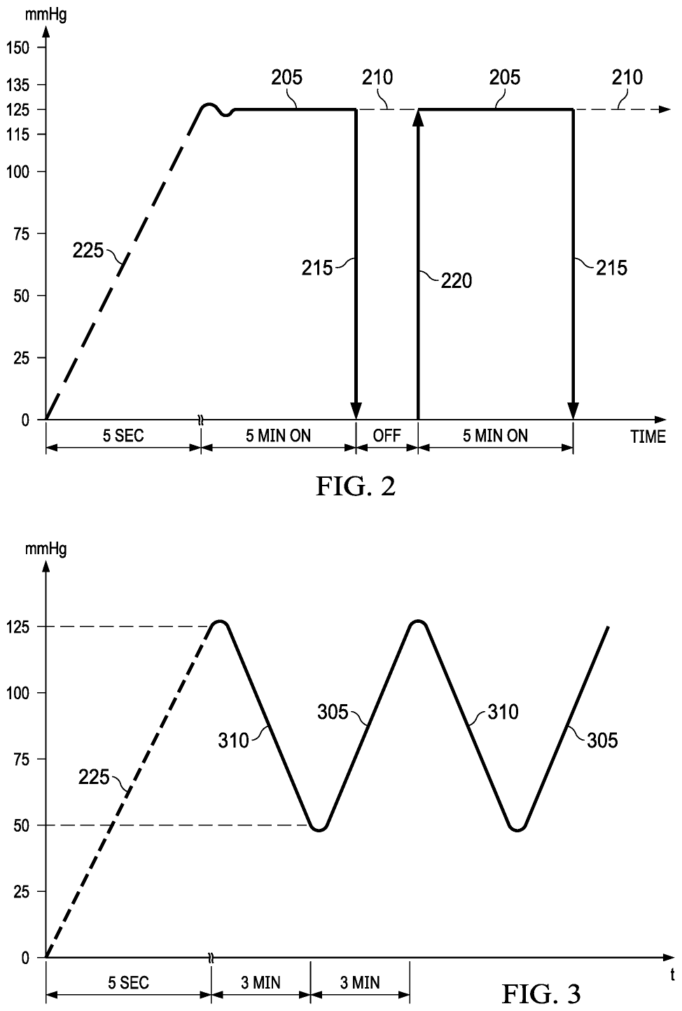

FIG. 2 is a graph illustrating additional details of an example control mode that may be associated with some embodiments of the controller 130. In some embodiments, the controller 130 may have a continuous pressure mode, in which the negative-pressure source 105 is operated to provide a constant target negative pressure, as indicated by line 205 and line 210, for the duration of treatment or until manually deactivated. Additionally or alternatively, the controller may have an intermittent pressure mode, as illustrated in the example of FIG. 2. In FIG. 2, the x-axis represents time and the y-axis represents negative pressure generated by the negative-pressure source 105 over time. In the example of FIG. 2, the controller 130 can operate the negative-pressure source 105 to cycle between a target pressure and atmospheric pressure. For example, the target pressure may be set at a value of 125 mmHg, as indicated by line 205, for a specified period of time (e.g., 5 min), followed by a specified period of time (e.g., 2 min) of deactivation, as indicated by the gap between the solid lines 215 and 220. The cycle can be repeated by activating the negative-pressure source 105, as indicated by line 220, which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time for negative-pressure, as indicated by the dashed line 225. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time, as indicated by the solid line 220, may be a value substantially equal to the initial rise time as indicated by the dashed line 225.

FIG. 3 is a graph illustrating additional details that may be associated with another example pressure control mode in some embodiments of the therapy system 100. In FIG. 3, the x-axis represents time and the y-axis represents negative pressure generated by the negative-pressure source 105. The target pressure in the example of FIG. 3 can vary with time in a dynamic pressure mode. For example, the target pressure may vary in the form of a triangular waveform, varying between a negative pressure of 50 and 125 mmHg with a rise time 305 set at a rate of +25 mmHg/min. and a descent time 310 set at −25 mmHg/min. In other embodiments of the therapy system 100, the triangular waveform may vary between negative pressure of 25 and 125 mmHg with a rise time 305 set at a rate of +30 mmHg/min and a descent time 310 set at −30 mmHg/min.

In some embodiments, the controller 130 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 130, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

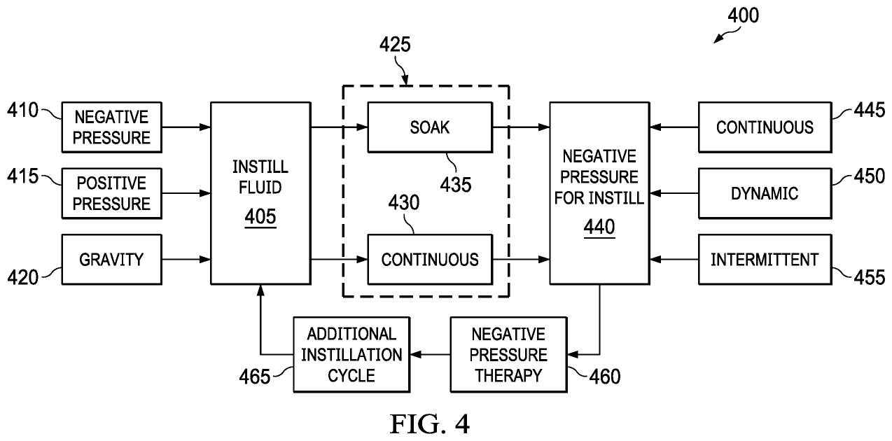

FIG. 4 is a chart illustrating details that may be associated with an example method 400 of operating the therapy system 100 to provide negative-pressure treatment and instillation treatment to the tissue interface 120. In some embodiments, the controller 130 may receive and process data, such as data related to instillation solution provided to the tissue interface 120. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second to 30 minutes. The controller 130 may also control the operation of one or more components of the therapy system 100 to instill solution, as indicated at 405. For example, the controller 130 may manage fluid distributed from the solution source 145 to the tissue interface 120. In some embodiments, fluid may be instilled to a tissue site by applying a negative pressure from the negative-pressure source 105 to reduce the pressure at the tissue site, drawing solution into the tissue interface 120, as indicated at 410. In some embodiments, solution may be instilled to a tissue site by applying a positive pressure from the positive-pressure source 150 to move solution from the solution source 145 to the tissue interface 120, as indicated at 415. Additionally or alternatively, the solution source 145 may be elevated to a height sufficient to allow gravity to move solution into the tissue interface 120, as indicated at 420.

The controller 130 may also control the fluid dynamics of instillation at 425 by providing a continuous flow of solution at 430 or an intermittent flow of solution at 435. Negative pressure may be applied to provide either continuous flow or intermittent flow of solution at 440. The application of negative pressure may be implemented to provide a continuous pressure mode of operation at 445 to achieve a continuous flow rate of instillation solution through the tissue interface 120, or it may be implemented to provide a dynamic pressure mode of operation at 450 to vary the flow rate of instillation solution through the tissue interface 120. Alternatively, the application of negative pressure may be implemented to provide an intermittent mode of operation at 455 to allow instillation solution to dwell at the tissue interface 120. In an intermittent mode, a specific fill volume and dwell time may be provided depending, for example, on the type of tissue site being treated and the type of dressing being utilized. After or during instillation of solution, negative-pressure treatment may be applied at 460. The controller 130 may be utilized to select a mode of operation and the duration of the negative pressure treatment before commencing another instillation cycle at 465 by instilling more solution at 405.

In addition to negative pressure wound therapy, a dressing disclosed herein may also be used as a secondary wound dressing for treating a tissue site.

Example Dressing

As noted above, the dressing 110 may comprise or consist essentially of a tissue interface 120, a cover 125, or both in some embodiments. The tissue interface 120 can be adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

Manifold

In some embodiments, the tissue interface 120 may comprise or consist essentially of one or more manifolds (also referred to as a manifold layer). A manifold or manifold layer may have a first side configured to be adjacent to a tissue site and a second side opposite to the first side. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed, or a secondary fluid path may be provided to facilitate delivering fluid, such as fluid from a source of instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a foam or other porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the manifold may form a manifold layer. The manifold layer may comprise a foam, such as a polymer foam. Non-limiting examples of a polymer foam as used herein comprise an acrylic, polyurethane, a polyolefin, a polyethylene, a polyacetate, a polyamide, a polyester, a polyether, a polyether block amide, a thermoplastic vulcanizate, a polyvinyl alcohol, or a combination thereof. In some embodiments, the manifold layer comprises a polyurethane ether foam. In further embodiments, the manifold layer may comprise an open-cell foam or a reticulated foam, or, more particularly, a reticulated polymer foam, such as a reticulated polyurethane foam. In some embodiments, the foam may be configured to form a tortuous path.

In some embodiments, a manifold may comprise or consist essentially of reticulated foam having pore sizes and free volume that may vary according to needs of a prescribed therapy. The tensile strength of the tissue interface 120 may also vary according to needs of a prescribed therapy. For example, the tensile strength of foam may be increased for instillation of topical treatment solutions. In some embodiments, a manifold may be foam comprised of polyols such as polyester or polyether, isocyanate such as toluene diisocyanate, and polymerization modifiers such as amines and tin compounds. In some examples, a manifold may be reticulated polyurethane foam such as found in GRANUFOAM™ dressing or V.A.C. VERAFLO™ dressing, both available from Kinetic Concepts, Inc. of San Antonio, Tex.

Other suitable materials useful in manifolds include non-woven fabrics (Libeltex, Freudenberg), three-dimensional (3D) polymeric structures (molded polymers, embossed and formed films, and fusion bonded films [Supracore]), and mesh, for example.

In some examples, a manifold may include a 3D textile, such as various textiles commercially available from Baltex, Muller, and Heathcoates. A 3D textile of polyester fibers may be particularly advantageous for some embodiments. For example, a manifold may comprise or consist essentially of a three-dimensional weave of polyester fibers. In some embodiments, the fibers may be elastic in at least two dimensions. A puncture-resistant fabric of polyester and cotton fibers having a weight of about 650 grams per square meter and a thickness of about 1-2 mm may be particularly advantageous for some embodiments. Such a puncture-resistant fabric may have a warp tensile strength of about 330-340 kilograms and a weft tensile strength of about 270-280 kilograms in some embodiments. Another particularly suitable material may be a polyester spacer fabric having a weight of about 470 grams per square meter, which may have a thickness of about 4-5 mm in some embodiments. Such a spacer fabric may have a compression strength of about 20-25 kilopascals (at 40% compression). Additionally or alternatively, a manifold may comprise or consist of a material having substantial linear stretch properties, such as a polyester spacer fabric having 2-way stretch and a weight of about 380 grams per square meter. A suitable spacer fabric may have a thickness of about 3-4 mm and/or may have a warp and weft tensile strength of about 30-40 kilograms in some embodiments. The fabric may have a close-woven layer of polyester on one or more opposing faces in some examples. In some embodiments, a woven layer may be advantageously disposed on a manifold to face a tissue site.

A manifold disclosed herein may be either hydrophobic or hydrophilic. In an example in which a manifold may be hydrophilic, the manifold may also wick fluid away from a tissue site, while continuing to distribute negative pressure to the tissue site. The wicking properties of a manifold may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In some embodiments, a manifold may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. A manifold may further serve as a scaffold for new cell-growth, or a scaffold material may be used in conjunction with a manifold to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials. Additional embodiments of manifolds for use in a dressing 110 are discussed further herein.

The thickness of a manifold may also vary according to needs of a prescribed therapy. For example, the thickness of a manifold may be decreased to reduce tension on peripheral tissue. The thickness of a manifold can also affect the conformability of the tissue interface 120. In some embodiments, a manifold thickness, e.g. for a suitable foam, may be in a range of about 2 mm to 10 mm, preferably about 2 mm to about 8 mm, more preferably about 3 mm to about 5 mm. Fabrics, including suitable 3D textiles and spacer fabrics, may also have a thickness in a range of about 2 mm to about 8 mm.

Felted Manifold

In some embodiments, the tissue interface 120 may comprise or consist essentially of a felted manifold or felted manifold layer that may comprise or consist essentially of felted foam. The felted foam or felted foam layer may serve as a manifold that may comprise interconnected pathways. Any suitable foam for felting may be used, including the example foams mentioned above.

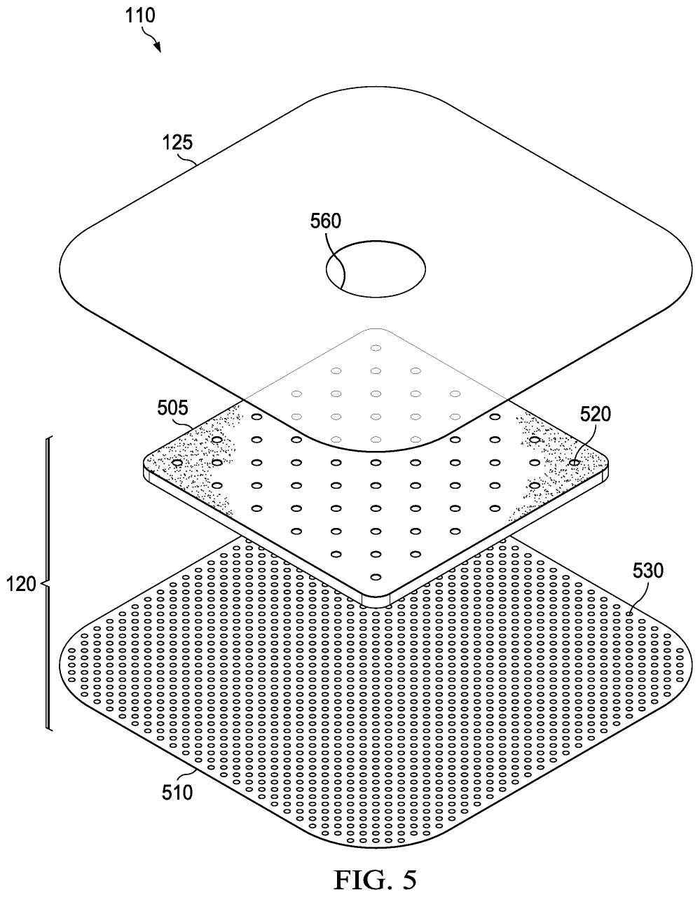

FIG. 5 is an assembly view of an example of the dressing 110 of FIG. 1, illustrating additional details that may be associated with some embodiments of the therapy system of FIG. 1. In some embodiments, the manifold 505 may be a felted foam.

In some embodiments, a compressed foam may also be referred to as a felted foam. A felted foam may undergo a thermoforming process to permanently compress the foam to increase the density of the foam. A felted foam may also be compared to other felted foams or compressed foams by comparing the firmness factor of the felted foam to the firmness factor of other compressed or uncompressed foams. A compressed or felted foam may have a firmness factor greater than 1.

Felting is a thermoforming process that permanently compresses a material. For example, in order to create felted foam, such as felted polyurethane, the foam is heated to an optimum forming temperature during the polyurethane manufacturing process and then it is compressed. The degree of compression controls the physical properties of the felted foam. For example, felted foam has an increased effective density and felting can affect fluid-to-foam interactions. As the density increases, compressibility or collapse decreases. Therefore, manifolds, such as various foams, which have different compressibility or collapse have different firmness values. The firmness of a felted manifold, e.g. felted foam is the felting ratio: original thickness/final thickness. In some example embodiments, a felted manifold “firmness” value or degree can range from about 1 to about 10, preferably about 2 to about 8, and more preferably from about 3 to about 7. In some embodiments, a felted manifold 505 may have a firmness factor (“FF”) of about 2, 3, 4, 5, 6, 7, 8, 9, 10, 15, 25, 50, 100 or any intermediate ranges or numbers. In particular embodiments, the felted manifold 505 may have a firmness factor of about five. For example, foam found in a GRANUFOAM™ dressing available from Kinetic Concepts, Inc. of San Antonio, Tex. may be felted to a density five times that of its uncompressed form. This would be referred to as firmness 5 felting. In another example, foam found in a GRANUFOAM™ dressing available from Kinetic Concepts, Inc. of San Antonio, Tex. may be felted to a density 4 times that of its uncompressed form. This would be referred to as firmness 4 felting. There is a general linear relationship between firmness level, density, pore size (or pores per inch) and compressibility under negative pressure. For example, foam found in a GRANUFOAM™ dressing that is felted to firmness 5 will not only show a five-fold density increase, but will only compress to about a fifth of its non-felted form.

A compressed foam may be a foam that is mechanically or chemically compressed to increase the density of the foam at ambient pressure. A compressed foam may be characterized by a firmness factor that is defined as a ratio of the density of a foam in a compressed state to the density of the same foam in an uncompressed state. The compressed state may refer to a state that is compressed by a force other than negative pressure, such as a mechanical force or a chemical force. For example, a firmness factor of 5 may refer to a compressed foam having a density that is five times greater than a density of the same foam in an uncompressed state. Mechanically or chemically compressing a foam may reduce a thickness of the foam at ambient pressure when compared to the same foam that has not been compressed. Reducing a thickness of a foam by mechanical or chemical compression may increase a density of the foam, which may increase the firmness factor of the foam. Increasing the firmness factor of a foam may increase a stiffness of the foam in a direction that is parallel to a thickness of the foam.

In some embodiments, a compressed foam may be a compressed V.A.C.® GRANUFOAM™ or similar foam. In some non-limiting embodiments, V.A.C.® GRANUFOAM™ may have a density of about 0.03 grams per centimeter³ (g/cm³) in its uncompressed state. If the V.A.C.® GRANUFOAM™ is compressed to have a firmness factor of 5, such V.A.C.® GRANUFOAM™ may be compressed until the density of the V.A.C.® GRANUFOAM™ is about 0.15 g/cm³. Additionally, V.A.C.® GRANUFOAM™ may have a thickness of 100 mm in its uncompressed state. If the V.A.C.® GRANUFOAM™ is compressed to have a firmness factor of 5, the V.A.C.® GRANUFOAM™ may be compressed until the thickness of the V.A.C.® GRANUFOAM™ is about 20 mm. V.A.C. VERAFLO™ foam may also be compressed to form a compressed foam having a firmness factor (FF) up to 5. The compressed foam, such as compressed V.A.C.® GRANUFOAM™ or V.A.C. VERAFLO™, may be further skived down to a manifold layer with a thickness of about 2 mm to about 8 mm, for example.

The manifold layer comprising a foam may have a pore size that varies according to needs of the tissue interface 120. For example, the manifold layer comprising a foam in its uncompressed state may have pore sizes in a range of about 400 microns to about 600 microns. The manifold layer comprising a foam in its compressed state, for a felted manifold, may, in some embodiments, have pore sizes smaller than 400 microns or less than 45 pore size per inch (ppi), determined by a measurement normal to the first side or second side of the foam. In some embodiments, the measurement is determined in the same direction as the direction of compression.

In certain embodiments, if a compressed foam is subjected to negative pressure, the compressed foam exhibits less deformation than a similar uncompressed foam. The decrease in deformation may be caused by the increased stiffness or density as reflected by the firmness factor (FF). If subjected to the stress of negative pressure, the compressed foam may flatten less than a similar uncompressed foam or may be less compressible than the similar uncompressed foam.

In some embodiments, the manifold 505 or manifold layer may comprise an absorbent material, for example, a super absorbent material. In particular embodiments, the manifold 505 may comprise a super absorbent, such as TEXSUS FP2325 or GELOK 30040-76 S/S/S absorbent. In further embodiments, the absorbent may be impregnated into the manifold 505. In some embodiments, the absorbent may be stable when dry and may swell and migrate out of the manifold 505 when wound exudate is introduced into the absorbent.

In an unsaturated state, the absorbent may have a first volume, which can be at least 5 percent less than the internal volume of an envelope inside the manifold 505 and can allow for free movement of fluids and distribution of pressure when positioned within the envelope. In some embodiments, the absorbent may have an unsaturated volume that is at least 10 percent less than the internal volume of the envelope. In some embodiments, the absorbent may have an unsaturated volume that is between 20 percent to about 90 percent of the internal volume of the envelope. In some embodiments, the envelope comprises a first wicking layer and a second wicking layer that may entirely surround or encapsulate absorbent. Further, in some embodiments, the absorbent may be moveable, expandable, or swellable within the envelope. For example, the absorbent may be configured to move, expand, or swell to a second volume if the absorbent becomes fully or partially saturated.

Biocompatible Polymer Compositions

In some embodiments, the manifold 505 or manifold layer may have a first side configured to be adjacent to the tissue site and a second side opposite to the first side, and a biocompatible polymer composition 520 present on the first side or the second side or both the first and the second sides of the manifold or manifold layer.

In some embodiments, the manifold 505 may be a felted foam at least partially or fully coated and/or printed with an active material, such as a biocompatible polymer composition 520 as illustrated in FIG. 5.

The biocompatible polymer composition 520 may comprise one or more structural proteins. Examples of a suitable structural protein may include, but are not limited to, collagen, keratin, fibronectin, fibrin, laminin, elastin, gelatin, and a mixture thereof. In particular embodiments, the structural protein comprises collagen. In various embodiments, the collagen may be obtained from any natural source. The collagen may be Type I, II, III or X collagen, or may also be chemically modified collagen, for example an atelocollagen obtained by removing the immunogenic telopeptides from natural collagen. The collagen may also comprise solubilized collagen or soluble collagen fragments, for example, having a molecular weight in the range of about 5,000 to about 100,000, or from about 5,000 to about 50,000. The solubilized collagen or soluble collagen fragments may be obtained by pepsin treatment of a natural collagen. In various embodiments, the collagen may be obtained from bovine corium that has been rendered largely free of non-collagenous components, for example, fat, non-collagenous proteins, polysaccharides, and other carbohydrates, as described in U.S. Pat. No. 4,614,794, Easton et al., issued Sep. 30, 1986 and U.S. Pat. No. 4,320,201, Berg et al., issued Mar. 16, 1982, each incorporated by reference herein in their entirety.

In some embodiments, the structural protein, such as collagen, may be present at a level of about 1% to about 90% by weight of the composition. In some more particular embodiments the composition comprises about 20 wt % to about 70 wt %, or about 40 wt % to about 65 wt %, or about 50 wt % to about 60 wt % structural protein, such as collagen, by weight of the composition.

Additionally the biocompatible polymer composition 520 may further comprise cellulose, such as oxidized regenerated cellulose (ORC) prepared by oxidation of a regenerated cellulose, such as rayon. The ORC may be manufactured by the process described in U.S. Pat. No. 3,122,479, which is incorporated herein by reference in its entirety. ORC is available with varying degrees of oxidation and hence rates of degradation. In some embodiments, the ORC may be in the form of water-soluble, low molecular weight fragments, for example, obtained by alkali hydrolysis of ORC.

In various embodiments, the ORC may be used in a variety of physical forms, including particles, fibers, a sheet, sponge, or fabrics. In some embodiments, the ORC is in the form of particles, such as fiber particles or powder particles, for example dispersed in a suitable solid or semisolid topical medicament vehicle. In some embodiments, the ORC comprises ORC fibers. In some, more particular embodiments, the ORC fibers may have a volume fraction such that at least 80% of the fibers have lengths in the range of about 5 μm to about 1000 μm, or in some more particular embodiments, about 250 μm to about 450 μm. In various embodiments, a desired size distribution can be achieved, for example, by milling an ORC cloth, followed by sieving the milled powder to remove fibers outside the range. Such fabrics may include woven, non-woven and knitted fabrics.

In some embodiments, the ORC may be present in the composition at a level of about 10% to about 98% by weight of the composition. In some, more particular embodiments, the composition comprises about 30% to about 95% or about 35% to about 70% ORC, by weight of the composition.

In some embodiments, the composition comprises a mixture of a structural protein, such as collagen, and ORC in a weight ratio of about 70:30 to about 30:70 or more particularly about 60:40 to about 40:60.

In some embodiments, the biocompatible polymer composition 520 can reduce bio-films and infections. In some embodiments, additional materials may be included in the biocompatible polymer composition 520. For example, additional materials such as antimicrobial agents, preservatives, stabilizing agents, plasticizers, matrix strengthening materials, dyestuffs, and combinations thereof may be present in the composition.

For example, in some embodiments, the biocompatible polymer composition 520 may also comprise one or more active materials, such as silver, citric acid, a non-steroidal anti-inflammatory drug (e.g. acetaminophen), a steroid, an antibiotic (e.g. penicillins or streptomycins), an antiseptic (e.g. chlorhexidine), and a growth factor (e.g. fibroblast growth factor or platelet derived growth factor). If present, such active material can be present at a level of about 0.1% to about 10%, or about 1% to about 5%, by weight of the composition.

In further embodiments, the biocompatible polymer composition 520 may further comprise a metal, for example silver, which may be used as an antimicrobial agent. The metal (e.g., silver) may be present in metallic form, in ionic form (e.g., a silver salt), or both. In some embodiments, silver may be present in combination with one or more additional metals, for example, gold, platinum, ferro-manganese, copper, zinc, or combinations thereof. The metal, particularly, silver, may confer antimicrobial properties to the dressing and in sufficiently lower concentrations, e.g., about 0.10 wt % to about 3.0 wt %, the silver may not cause cytotoxicity in a wound or at a tissue site. The term “tissue site” may refer to areas of any tissue that are not necessarily wounded or defective, but are instead areas in which it may be desirable to add or promote the growth of additional tissue.

In some embodiments, at least a portion of the metal may be present as a complex of ORC and the metal, for example, as an ORC-silver complex. As used herein, the term “complex” refers to an intimate mixture at the molecular scale, preferably with ionic or covalent bonding between the metal (e.g., silver) and the polysaccharide (e.g., ORC). The complex may comprise a salt formed between the anionic polysaccharide and Ag+, but it may also comprise silver clusters and/or colloidal silver metal, for example produced by exposure of the complex to light. For example, an anionic polysaccharide (e.g., ORC) may be treated with a silver salt solution to produce a complex of the anionic polysaccharide (e.g., ORC) with silver. The silver salt solution may be an aqueous solution and the solution may be prepared in a quantity sufficient to provide the desired silver concentration in the resultant complex. In some embodiments, the amount of silver in the complex may be about 0.1% to about 50% by weight based on the weight of the anionic polysaccharide, particularly, about 1% to about 40%, about 2% to about 30% by weight, and about 5% to about 25% by weight.

In various embodiments, an OCR-metal complex (e.g., ORC-silver complex) may be present in an amount ≥about 0.10 wt %, ≥about 0.50 wt %, ≥about 1.0 wt %, ≥about 2.0 wt %, ≥about 3.0 wt %, ≥about 4.0 wt %, ≥about 5.0 wt %, ≥about 6.0 wt %, ≥about 8.0 wt %, or ≥about 10 wt %. Additionally or alternatively, an ORC-metal complex (e.g., ORC-silver complex) may be present based on the total weight of the composition, in amount of about 0.10 wt % to about 10 wt %, about 0.10 wt % to about 8.0 wt %, about 0.10 wt % to about 5.0 wt %, about 0.50 wt % to about 4.0 wt %, about 0.50 wt % to about 3.0 wt %, or about 0.50 wt % to about 2.0 wt %.

In some embodiments, the biocompatible polymer composition 520 may include a carrier to dissolve, soften, and/or promote plasticity of a biocompatible polymer, such as collagen and/or ORC, to form the biocompatible polymer composition 520. The biocompatible polymer composition 520 can then be applied to the manifold 505, and the biocompatible polymer, such as collagen and/or ORC, may be released when, for example, water from wound exudate or water fluid reach the carrier.

Cover

As illustrated in FIGS. 1 and 5, in addition to the tissue interface 120, the dressing 110 may further include the cover 125. As illustrated in FIG. 5, the cover 125 may comprise a fluid communication channel 560 configured to distribute negative pressure evenly. In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a non-porous polymer drape or film, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Coveris Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

Attachment Device/Adhesive

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

Fluid Control Layer

As illustrated in FIG. 5, in some embodiments, the tissue interface 120 may further comprise, in addition to one or more manifolds, a fluid control layer 510 adjacent to the one or more manifolds and disposed between the one or more manifolds and the tissue site. The fluid control layer 510 may comprise or consist essentially of a means for controlling or managing fluid flow. In some embodiments, the fluid control layer 510 may be a polymer film comprising or consisting essentially of a liquid-impermeable, elastomeric material. For example, the polymer film may comprise or consist essentially of a polyurethane film. In some embodiments, the polymer film may comprise or consist essentially of the same material as the cover 125. The polymer film may also have a smooth or matte surface texture in some embodiments. A glossy or shiny finish better or equal to a grade B3 according to the SPI (Society of the Plastics Industry) standards may be particularly advantageous for some applications. In some embodiments, variations in surface height may be limited to acceptable tolerances. For example, the surface of the polymer film may have a substantially flat surface, with height variations limited to 0.2 mm over a cm.

In some embodiments, the polymer film may be hydrophobic. The hydrophobicity of the polymer film may vary, but may have a contact angle with water of at least ninety degrees in some embodiments. In some embodiments the polymer film may have a contact angle with water of no more than 150 degrees. For example, in some embodiments, the contact angle of the polymer film may be in a range of at least 90 degrees to about 120 degrees, or in a range of at least 120 degrees to 150 degrees. Water contact angles can be measured using any standard apparatus. Although manual goniometers can be used to visually approximate contact angles, contact angle measuring instruments can often include an integrated system involving a level stage, liquid dropper such as a syringe, camera, and software designed to calculate contact angles more accurately and precisely, among other things. Non-limiting examples of such integrated systems may include the FTÅ125, FTÅ200, FTÅ2000, and FTÅ4000 systems, all commercially available from First Ten Angstroms, Inc., of Portsmouth, Va., and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles herein represent averages of 5-9 measured values, discarding both the highest and lowest measured values. The hydrophobicity of the polymer film may be further enhanced with a hydrophobic coating of other materials, such as silicones and fluorocarbons, either as coated from a liquid, or plasma coated.

The polymer film may also be suitable for welding to other layers, including to the one or more manifolds. For example, the polymer film may be adapted for welding to polyurethane foams using heat, radio frequency (RF) welding, or other methods to generate heat such as ultrasonic welding. RF welding may be particularly suitable for more polar materials, such as polyurethane, polyamides, polyesters and acrylates. Sacrificial polar interfaces may be used to facilitate RF welding of less polar film materials, such as polyethylene.

The area density of the polymer film may vary according to a prescribed therapy or application. In some embodiments, an area density of less than 40 grams per square meter may be suitable, and an area density of about 20-30 grams per square meter may be particularly advantageous for some applications.

In some embodiments, for example, the polymer film may comprise or consist essentially of a hydrophobic polymer, such as a polyethylene film. The simple and inert structure of polyethylene can provide a surface that interacts little, if any, with biological tissues and fluids, providing a surface that may encourage the free flow of liquids and low adherence, which can be particularly advantageous for many applications. Other suitable polymeric films include polyurethanes, acrylics, polyolefin (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate, styreneics, silicones, fluoropolymers, and acetates. A thickness between 20 microns and 100 microns may be suitable for many applications. Films may be clear, colored, or printed. More polar films suitable for laminating to a polyethylene film include polyamide, co-polyesters, ionomers, and acrylics. To aid in the bond between a polyethylene and polar film, tie layers may be used, such as ethylene vinyl acetate, or modified polyurethanes. An ethyl methyl acrylate (EMA) film may also have suitable hydrophobic and welding properties for some configurations.

Additionally, the polymer film may have one or more fluid restrictions 530, which can be distributed uniformly or randomly across the polymer film. The fluid restrictions 530 may be bi-directional and pressure-responsive. For example, each of the fluid restrictions 530 generally may comprise or consist essentially of an elastic passage that is normally unstrained to substantially reduce liquid flow, and can expand or open in response to a pressure gradient. In some embodiments, the fluid restrictions 530 may comprise or consist essentially of perforations in the polymer film. Perforations may have a uniform size or vary in size. Perforations may be formed by removing material from the polymer film. For example, perforations may be formed by cutting through the polymer film, which may also deform the edges of the perforations in some embodiments. In the absence of a pressure gradient across the perforations, the passages may be sufficiently small to form a seal or fluid restriction, which can substantially reduce or prevent liquid flow. Additionally or alternatively, one or more of the fluid restrictions 530 may be an elastomeric valve that is normally closed when unstrained to substantially prevent liquid flow, and can open in response to a pressure gradient. A fenestration in the polymer film may be a suitable valve for some applications. Fenestrations may also be formed by removing material from the polymer film, but the amount of material removed and the resulting dimensions of the fenestrations may be up to an order of magnitude less than perforations, and may in some instances not deform the edges.

For example, some embodiments of the fluid restrictions 530 may comprise or consist essentially of one or more slits, slots or combinations of slits and slots in the polymer film. In some examples, the fluid restrictions 530 may comprise or consist of linear slots having a length less than 4 mm and a width less than 1 mm. The length may be at least 2 mm, and the width may be at least 0.4 mm in some embodiments. A length of about 3 mm and a width of about 0.8 mm may be particularly suitable for many applications, and a tolerance of about 0.1 mm may also be acceptable. Such dimensions and tolerances may be achieved with a laser cutter, for example. Slots of such configurations may function as imperfect valves that substantially reduce liquid flow in a normally closed or resting state. For example, such slots may form a flow restriction without being completely closed or sealed. The slots can expand or open wider in response to a pressure gradient to allow increased liquid flow.

Example Methods of Making

Also disclosed herein are methods of making the dressing 110 comprising a tissue interface 120. In various embodiments, methods comprise providing a porous open-cell liquid-permeable felted foam or a fluid permeable material, and applying the polymer composition or the biocompatible polymer composition to the foam or fluid permeable material to attach the polymer composition or the biocompatible polymer to the foam or fluid permeable material. The providing may comprise compressing a foam having a firmness less than (and thickness greater than) the felted foam

In some embodiments, the methods comprise felting at least one manifold, for example a foam, to a desired degree of firmness, for example a firmness factor of five. As discussed above, felting is a thermoforming process whereby material, such as foam, is permanently compressed.

In some example embodiments, at least one manifold, such as a foam, particularly an open-celled polymeric foam, may be felted by heating, for example, to approximately 150° C. The manifold may then be compressed down with an appropriate force or weight to achieve a desired firmness factor or compression level. For example, to achieve a compression level or firmness factor of five, the manifold may be permanently compressed from 100 mm thick to 20 mm thick.

In some further example embodiments, the foam 505, such as the felted foam, may be skived down. For example, the manifold may be skived down from a thickness of about 20 mm to a thickness of about 2 to about 8 mm thick. The resulting foam may have a felted highly concentrated open-cell structure with highly concentrated struts and a reduced size of the over pore structure. The foam may be configured to form a tortuous path that enables the manifold to hold onto the composition comprising a biocompatible polymer, such as collagen and ORC. The foam may also be configured to gradually deliver the composition as the composition is solubilized by the wound fluid from the tissue site.

In further example embodiments, the methods to make the tissue interface 120 may further comprise applying the biocompatible polymer composition 520 to the manifold 505. The biocompatible polymer composition 520 may be deposited discretely to the manifold 505 by any methods known in the art, for example, printing techniques or coating techniques. The coating techniques may comprise pattern-coating, deposition-coating or plasma coating, or in some embodiments, coating the manifold 505 with a solution comprising a solvent and the biocompatible polymer composition 520 and drying the solvent. For example, adhesives such as glues may be deposited using 3 axis printers where positive pressure pumps or air pressures are used to force the adhesives through nozzles and onto a substrate, such as the manifold 505. In some embodiments, when the biocompatible polymer composition 520 comprises collagen and ORC, the collagen and ORC may be formed into a slurry where the dry materials, such as collagen and ORC, are ground into a particulate and then dispersed in a suitable carrier. In some example embodiments, the carrier may be a water soluble or water-sensitive polymer. In various embodiments, a “water-soluble” polymer is a material having a solubility in water of 10 mg/L and greater at standard temperature and pressure. In various embodiments, a “water-sensitive” polymer is a material that undergoes a physical or chemical change when contacted with water. For example, polymers useful as carriers may include polyvinyl alcohol or polyvinylpyrrolidone (PVP). In some embodiments, the carrier may be a carboxyl substituted polymer which is dissolved in water or an organic solvent such as isopropyl alcohol. In these embodiments, the solvents may be subsequently evaporated to leave the collagen and ORC attached or glued to the substrate, such as the manifold 505. In further embodiments, the carrier may be softened by the addition of plasticizers, such as glycerol and polyethylene glycols.

Kits

Also disclosed herein are wound therapy kits comprising a dressing 110 comprising a tissue interface 120 described herein. A wound therapy kit may comprise multiple components which may or may not be co-packaged together. The wound therapy kits may comprise two or more manifolds having different firmness, optionally having a fenestrated polymer film laminated thereon. In some embodiments, at least one of the manifolds is felted, such as a felted foam described herein. The kits may further comprise one or more covers, such as a drape; and one or more dressing interfaces, such as a SENSAT.R.A.C.™ Pad available from Kinetic Concepts, Inc. of San Antonio, Tex. End users may be able to use the wound therapy kit to customize the tissue interface 120 (e.g. a wound filler) for the dressings described herein for use during negative-pressure therapy.

### Additional or Alternative Embodiments

Additionally or alternatively, the manifold 505 may further comprise a wicking layer. Additionally or alternatively, the manifold 505 may be plasma or corona treated to increase the hydrophilicity of the manifold 505 to drive the exchange of wound fluid with the deposited composition. Additionally or alternatively, the biocompatible polymer composition 520 may be printed onto an absorbent foam such as AMS (Advanced Medical Systems) MCF03 or Freudenberg Hydrophilic PUC Foam—1034. For example, the absorbent foam may be between about 3 mm and about 5 mm in thickness and encapsulated between a fluid control layer 510 and a cover 125.

### Advantages

The compositions, dressings, systems, and the methods described herein may provide significant advantages. For example, the dressing described herein can facilitate the easy delivery of a biocompatible polymer composition 520, for example comprising collagen and ORC, to reduce the effect of delayed wound healing. Without being bound by theory, a felted foam described herein is capable of acting as a manifolding layer that has highly concentrated struts and a reduced pore size. This reduced and concentrated structure forms a tortuous path that enables the manifold to retain the biocompatible polymer composition 520, e.g. comprising collagen and ORC, and only gradually deliver the biocompatible polymer composition 520, e.g. as the collagen and ORC is solubilized by the wound fluid. Additionally, the felted foam can provide a porous manifold to effectively distribute negative pressure to the tissue site and will not substantially swell when in contact with water so the dressing can maintain good pressure transfer in use.

### Non-limiting Discussion of Terminology

The description and specific examples above, while providing illustrative embodiments of the claimed subject matter, are intended for purposes of illustration only and are not intended to limit the scope of the claimed subject matter. Moreover, recitation of multiple embodiments having stated features is not intended to exclude other embodiments having additional features, or other embodiments incorporating different combinations of the stated features. Components may be also be combined or eliminated in various configurations for purposes of sale, manufacture, assembly, or use. Specific examples are provided for illustrative purposes of how to make and use the claimed subject matter and, unless explicitly stated otherwise, are not intended to be a representation that given embodiments have, or have not, been made or tested. Equivalent changes, modifications and variations of some embodiments, materials, compositions and methods can be made within the scope of the appended claims with substantially similar results.

As used herein, the word “include,” and its variants, is intended to be non-limiting, such that recitation of items in a list is not to the exclusion of other like items that may also be useful in the materials, compositions, devices, and methods of the claimed subject matter. Similarly, the terms “can” and “may” and their variants are intended to be non-limiting, such that recitation that an embodiment can or may comprise certain elements or features does not exclude other embodiments that do not contain those elements or features. Moreover, descriptions of various alternatives using terms such as “or” do not require mutual exclusivity unless clearly required by the context, and the indefinite articles “a” or “an” do not limit the subject to a single instance unless clearly required by the context.

Although the open-ended term “comprising,” as a synonym of non-restrictive terms such as including, containing, or having, is used herein to describe and claim certain embodiments, embodiments may alternatively be described using more limiting terms such as “consisting of” or “consisting essentially of.” Thus, for any given embodiment reciting materials, components or process steps, the claimed subject matter may also specifically include embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosure of values and ranges of values for specific parameters (such as temperatures, molecular weights, weight percentages, etc.) are not exclusive of other values and ranges of values useful herein. Two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

Unless indicated otherwise, all numeric values are to be interpreted to be “about” such values. The term “about” is intended to refer to deviations in a numerical quantity that may result from various circumstances, for example, through measuring or handling procedures in the real world; through inadvertent error in such procedures; through differences in the manufacture, source, or purity of compositions or reagents; from computational or rounding procedures; and other deviations as will be apparent by those of skill in the art from the context of the example embodiments. For example, the term “about” may refer to deviations that are greater or lesser than a stated value or range by 1/10 of the stated value(s), e.g., ±10%, as appropriate from the context of the examples. For instance, a concentration value of “about 30%” may refer to a concentration between 27% and 33%. Whether or not modified by the term “about,” quantitative values recited in the claims include equivalents to the recited values, for example, deviations from the numerical quantity, as would be recognized as equivalent by a person skilled in the art.

The appended claims set forth novel and inventive aspects of the subject matter disclosed and described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

1. A dressing for treating a tissue site, the dressing comprising:
a manifold layer comprising:
a porous open-cell liquid-permeable foam comprising 50 to 150 micron sized pores capable of distributing negative pressure to the tissue site and withdrawing tissue exudate; and
a polymer composition bound to the foam, the polymer composition comprising an active agent and a polymer carrier for the active agent, the polymer carrier capable of releasing the active agent when exposed to tissue exudate; and
wherein the foam has a first side configured to be adjacent to the tissue site and a second side opposite to the first side, and the polymer composition is present on the first side or the second side or both the first and the second sides of the foam, and
wherein the pore size is determined by a measurement normal to the first side or second side of the foam.

2. The dressing of claim 1, wherein the foam has more than 350 pores per linear inch (ppi) as determined by a measurement normal to the first side or second side of the foam.

3. The dressing of claim 1, wherein the manifold layer has a thickness of 2 mm to 8 mm.

4. (canceled)

5. The dressing of claim 1, wherein the foam comprises a felted foam.

6. (canceled)

7. The dressing of claim 1, wherein the foam comprises a polymer foam selected from the group consisting of acrylic, polyurethane, polyolefin, polyethylene, polyacetate, a polyamide, polyester, polyether, polyether block amide, thermoplastic vulcanizate, and polyvinyl alcohol foams, and combinations thereof.

8. (canceled)

9. The dressing of claim 1, wherein at least a portion of the foam is a plasma or corona treated foam that increases the hydrophilicity of the treated portion of the foam as compared to the same foam that has not been treated.

10. (canceled)

11. The dressing of claim 1, wherein the foam comprises a reticulated polymer foam.

12. (canceled)

13. The dressing of claim 1, wherein the foam has a firmness factor of about 5.

14. The dressing of claim 1, wherein the manifold layer comprises pores having a tortuous path between the first side and the second side.

15. The dressing of claim 1, wherein the manifold layer further comprises an absorbent.

16. (canceled)

17. The dressing of claim 1, further comprising an adhesive coupling the polymer composition to the manifold layer.

18. The dressing of claim 1, wherein the active agent comprises collagen, oxidized regenerated cellulose (ORC), or both collagen and ORC.

19. (canceled)

20. (canceled)

21. The dressing of claim 1, wherein the polymer carrier comprises a water-soluble and/or water-sensitive polymer.

22. The dressing of claim 21, wherein the polymer carrier comprises polyvinyl alcohol, polyvinylpyrrolidone (PVP), or a carboxyl-substituted polymer.

23. The dressing of claim 1, wherein the dressing further comprises a cover configured to be disposed adjacent to the manifold layer and to form a seal around the tissue site.

24. (canceled)

25. The dressing of claim 1, wherein the dressing further comprises a fluid-control layer configured to be disposed between the manifold layer and the tissue site, wherein the fluid-control layer has a plurality of fluid restrictions.

26. The dressing of claim 25, wherein the fluid-control layer comprises a polymer film.

27. (canceled)

28. (canceled)

29. (canceled)

30. The dressing of claim 26, wherein the polymer film is a polyethylene film having an area density of less than 30 grams per square meter.

31. (canceled)

32. (canceled)

33. (canceled)

34. The dressing of claim 25, wherein the fluid restrictions comprise a plurality of perforations configured to permit fluid flow and inhibit exposure of the manifold layer to the tissue site.

35. The dressing of claim 25, wherein the fluid restrictions comprise a plurality of slits or slots, each of the slits or slots having a length less than 4 millimeters.

36. The dressing of claim 25, wherein the fluid restrictions comprise elastomeric valves in the fluid-control layer.

37. (canceled)

38. (canceled)

39. (canceled)

40. (canceled)

41. The dressing of claim 1, wherein the dressing further comprises a wicking material in contact with the foam.

42. The dressing of claim 41, wherein the wicking material is capable of driving the exchange of tissue exudate with the active agent.

43. A dressing for treating a tissue site, the dressing comprising:
a fluid permeable material comprising a plurality of pores having a first surface, a second surface, and a third surface extending between the first and second surfaces, wherein each of the plurality of pores has a pore size in at least one dimension at the third surface permanently smaller than the diameter of the pore at the plane of the first and/or second surface; and
a biocompatible polymer composition adhered to the fluid permeable material, the composition comprising collagen, oxidized regenerated cellulose (ORC), and a water-soluble and/or water-sensitive polymer.

44. (canceled)

45. (canceled)

46. (canceled)

47. (canceled)

48. A method of treating a tissue site with negative pressure, the method comprising:
applying the dressing of claim 1 to a tissue site;
sealing the dressing in a void adjacent to the tissue site, wherein the sealing is configured to allow the dressing to provide negative pressure to the tissue site;
fluidly coupling the dressing to a negative-pressure source; and
applying negative pressure from the negative-pressure source to the dressing.

49. (canceled)

50. (canceled)

51. (canceled)

52. The method of claim 48, further comprising delivering a fluid from a fluid source through the dressing.

53. The method of claim 52, wherein the method provides collagen, oxidized regenerated cellulose (ORC), and/or another active agent to the tissue site.

54.-74. (canceled)
